(19) United States
(12) Patent Application Publication
(10) Pub. No.: US 2015/0075139 A1
(43) Pub. Date: Mar. 19, 2015

(54) METHOD FOR CONTROLLING AN INJECTOR PLACED IN A MOTOR VEHICLE EXHAUST LINE

(71) Applicants: Brice HEMBERT, ARNAUD-GUILHEM (FR); Steven MAERTENS, TOULOUSE (FR); Nic VAN VUUREN, YORKTOWN, VA (US)

(72) Inventors: Brice HEMBERT, ARNAUD-GUILHEM (FR); Steven MAERTENS, TOULOUSE (FR); Nic VAN VUUREN, YORKTOWN, VA (US)

(73) Assignees: CONTINENTAL AUTOMOTIVE GmbH, Hannover (DE); CONTINENTAL AUTOMOTIVE FRANCE, Toulouse (FR)

(21) Appl. No.: 14/485,908

(22) Filed: Sep. 15, 2014

(30) Foreign Application Priority Data
Sep. 19, 2013 (FR) 1359037

(51) Int. Cl. F01N 3/20; F01N 9/00; G01N 33/00

(52) U.S. Cl. CPC F01N 3/208; G01N 33/0037; G01N 33/0009; F01N 9/00
USPC 60/274

(57) ABSTRACT

A method for controlling an injector (1) placed in a motor vehicle exhaust line (2) for the metered injection of a liquid, such as notably a reducing agent, includes commanding openings of the injector for lengths of time that are suited to ensure that the flow of liquid delivered upon each of the openings consists in a drop of liquid by commanding constant injector-opening times, and adjusting the metered dose of liquid injected by varying the frequency of such openings.

### FIELD OF THE INVENTION

The invention relates to a method for controlling an injector placed in a motor vehicle exhaust line for the metered injection of a liquid, such as notably a reducing agent.

### BACKGROUND OF THE INVENTION

Selective catalytic reduction is currently one of the most effective techniques for “cleaning up” diesel engines. This technique involving the post-treatment of the exhaust gases uses solutions based on ammonia or on urea to break down the oxides of nitrogen (NOₓ) into diatomic nitrogen (N₂) and water vapor: these solutions are injected into the exhaust line and cause a chemical reaction within the catalytic converter following which reaction the potentially harmful exhaust gases are converted into water vapor and into nitrogen which is not harmful to the environment.

Such an injection technique does, however, prove to be a potential source of malfunctioning when, notably when the engine is operating at high load, during active-regeneration phases, etc., the temperature of the exhaust gases reaches high temperatures, of the order of 500° C. to 700° C. near the injection nozzle of the injectors. This is because such temperatures may:
impair the thermodynamic integrity of the injector,
cause the creation of residues likely to at least partially block the injection nozzle, lead to accelerated corrosion.

As the consequences of such detrimental effects are highly damaging, numerous studies have been conducted aimed at solving this problem.

Following these studies, the technique most widely used at the present time is, when high exhaust gas temperatures are detected:
when the engine is running at normal or high load, to command the injection of a flow of liquid that is suited to removing heat energy by convection and, at the same time, to preventing the creation and/or depositing of residues and avoiding any corrosion phenomenon,
when the engine returns to its low-idle speed, to increase the flow of liquid injected so as to prevent the period of “overheating” of the injector that occurs after the engine has stopped,
when the engine has stopped, to command, generally after a phase of waiting for the exhaust gases to cool down, a purge that involves reversing the direction of flow of the liquid so as to suck up this liquid and the exhaust gases and thus prevent the creation and/or depositing of residues and avoid any corrosion phenomenon. It should be noted that the previous solution (injection of liquid) is not recommended after the engine has stopped because of the risk of an accumulation of deposits in the exhaust line.

However, such a technique still has disadvantages. Specifically, the solution employed when the engine is running leads to a high consumption of liquid, with all the consequences inherent to such additional consumption: cost, high volume of liquid carried on board and therefore high on-board weight, or high filling frequency, etc.

The solution employed when the engine is stopped is able, for its part, only to prevent the creation and/or the depositing of residues and to avoid any corrosion phenomenon. By contrast, it has no effect of cooling the injector nozzle. In addition, the solution proves to be very expensive because it requires, on the one hand, an injection pump provided with a reverser, or two pumps one dedicated to injection and one dedicated to purge, and, on the other hand, means for controlling and managing the reversal or the switchover.

Numerous other solutions have been developed with a view to addressing these disadvantages, and these notably include:
changing the structure or the material with a view to encouraging the cooling of the injector (nozzle made of graphite, cooling fins, etc.),
injector cooling circuit using engine cooling water,
double-wall exhaust line delimiting a peripheral pipe forming a water-filled closed volume,
recirculating at the injector some of the liquid delivered to this injector, allowing high flows of liquid.

However, all of these solutions lead to appreciable additional costs which are practically unacceptable within the automotive sector.

### SUMMARY OF THE INVENTION

The present invention seeks to alleviate all of the disadvantages of the current solutions described hereinabove aimed at protecting the injectors placed in the exhaust lines, and the essential objective of the present invention is to provide a workable solution that gives rise neither to additional consumption of liquid nor to additional equipment cost.

To this end, the invention envisions a method for controlling an injector placed in a motor vehicle exhaust line for the metered injection of a liquid, such as notably a reducing agent, which, according to the invention, consists in commanding openings of said injector for lengths of time that are suited to ensuring that the flow of liquid delivered upon each of the openings consists in a drop of liquid by commanding constant injector-opening times and adjusting the metered dose of liquid injected by varying the frequency of such openings.

According to this method, the liquid is therefore injected drop by drop by commanding frequent openings for very short lengths of time, so as to remain within an injector-opening zone that does not allow a fully formed jet of liquid to be created.

Such a method, which is very simple to implement, therefore leads to the formation on the tip of the injector of drops of liquid the water of which is made to evaporate at this tip and the urea of which breaks down into NH3 and, in practice, the creation of these drops of water has a threefold effect:
heat energy is removed during the phase of conversion from fluid to vapor (latent heat)
the risk of residue formation is reduced because of the low “drop-by-drop” flow of liquid,
the risk of corrosion is reduced.

The method according to the invention therefore, first of all, in order to implement it, requires a conventional injector of the current type and simply involves controlling this injector in such a way that the metered dose of liquid is injected “drop by drop” by commanding very short opening times for this injector that correspond to its operating in an injector-opening zone that does not allow a fully formed jet of liquid to be created.

In addition, the injector-opening time is advantageously substantially of the order of 1 millisecond.

Moreover, when a motor vehicle stops, the procedure of injecting liquid drop by drop is, advantageously according to the invention, continued for a predetermined period of time following this stopping.

This procedure of injecting after the engine has stopped can be substituted for the purge procedure and, at the very least, it leads to a removal of heat energy and to a reduction of the risk of the formation of residues and the risk of corrosion while it is being implemented, which is generally during the waiting phase that precedes the purge operation.

In addition, implementation of the injection procedure according to the invention after the engine has stopped makes it possible to reduce the flow of liquid injected during the phase preceding this stopping of the engine.

### BRIEF DESCRIPTION OF THE DRAWINGS

Further features, objects and advantages of the invention will become apparent from the detailed description which follows with reference to the attached drawings which by way of nonlimiting example depict one preferred embodiment thereof. In these drawings:

FIG. 1 is a schematic view of a selective catalytic reduction installation allowing implementation of the method according to the invention,

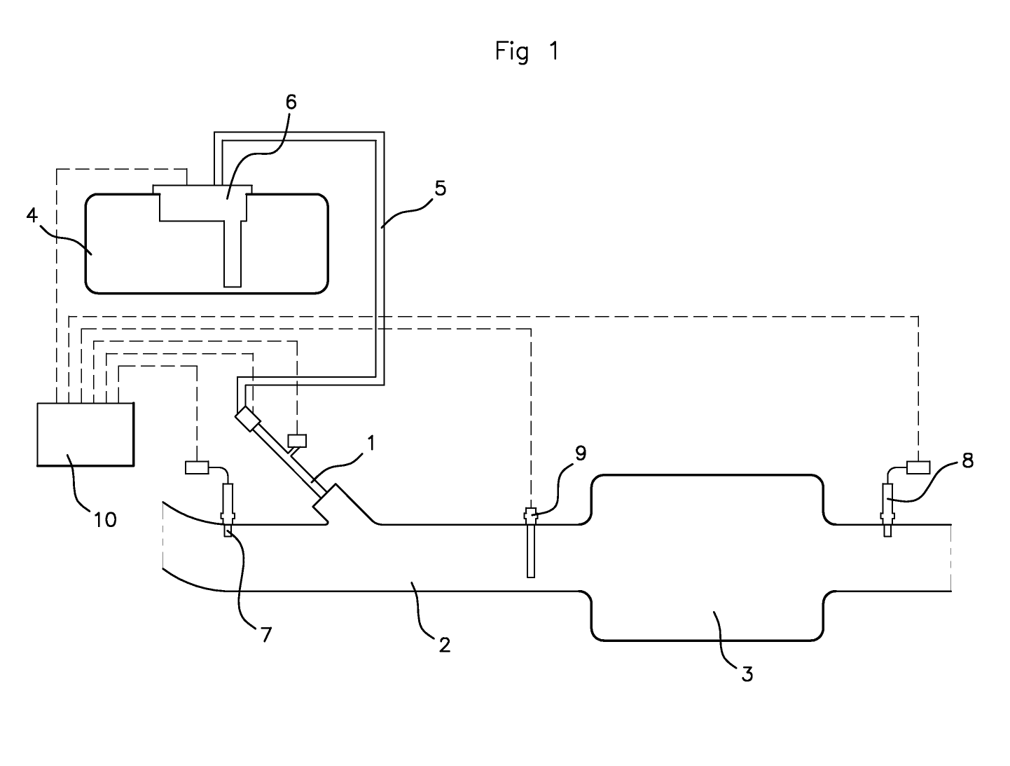

FIG. 2 is a diagram depicting the operating curve of an injector.

### DETAILED DESCRIPTION OF THE INVENTION

The method according to the invention seeks to control an injector 1 placed in an exhaust line 2 of a motor vehicle fitted with a selective catalytic reduction installation comprising, first of all, in addition to said injector 1, a selective catalytic reduction catalytic converter 3 positioned downstream of this injector 1.

This installation also comprises a reservoir 4 containing the liquid that is to be injected which consists in a solution based on urea and on water, connected to the injector 1 by a pipe 5 in which the liquid is distributed using a pump 6.

This installation also comprises a plurality of sensors chiefly consisting of:
a sensor 7 that measures the level of nitrogen oxides present in the exhaust gases upstream of the injector 1,
a sensor 8 that measures the level of nitrogen oxides present in the exhaust gases at the outlet of the catalytic converter 3,
and a sensor 9 that measures the temperature of the exhaust gases at the inlet to the catalytic converter 3.

These various sensors 7-9 are connected to a central control unit 10 that controls the catalytic reduction installation and is programmed notably to determine the metered amount of liquid to be injected and, according to the invention, to control the injector 1 in such a way that this metered dose is delivered drop by drop by commanding injector-opening times substantially of the order of 1 millisecond.

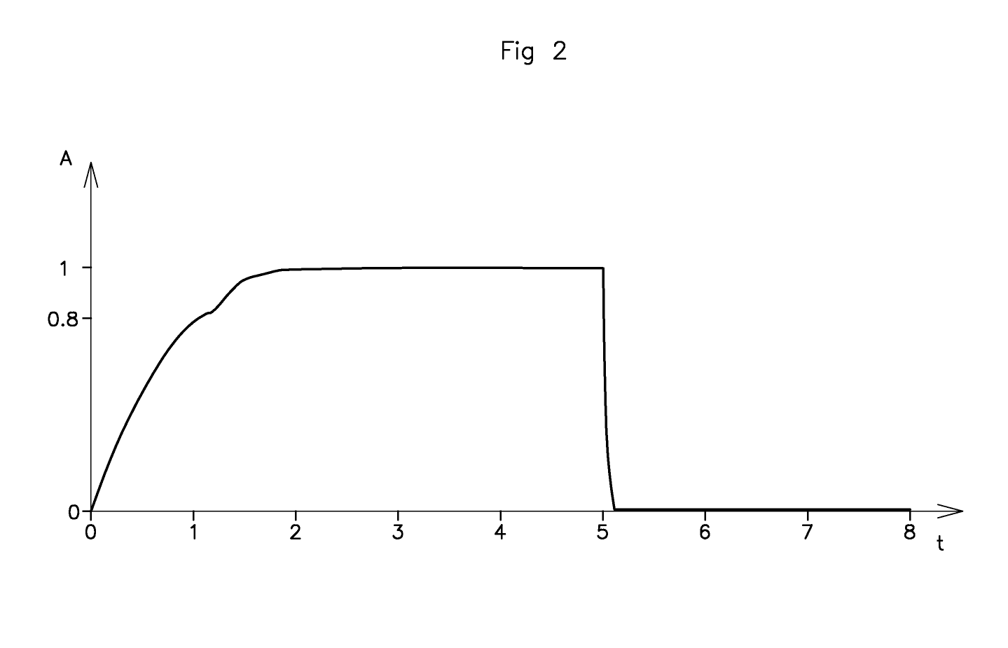

As depicted in FIG. 2 which depicts the operating curve of an injector 1 as a function of the strength of the current supplied to said injector, this duration of 1 ms corresponds to a partial opening of the injector 1 that does not allow a fully formed jet of liquid to be created.

The procedure of injecting “drop by drop” according to the invention allows the oxides of nitrogen NOₓ to be broken down effectively into diatomic nitrogen N₂ and water vapor, while at the same time causing a removal of heat energy ensuring the thermodynamic integrity of the injector, and reducing the risks of the formation of residues and the risks of corrosion.

Such a procedure of injecting “drop by drop” is intended, while engines are running, to take the place of the current injection procedures possibly with ad hoc increases in the metered doses delivered when, notably, the temperature and/or nitrogen oxides levels require this.

This procedure of injecting “drop by drop” is also intended to be implemented after the engine has stopped, for example during the phase of waiting for the purge, so as to remove heat energy and reduce the risks of the formation of residues and the risks of corrosion.

1. A method for controlling an injector (1) placed in a motor vehicle exhaust line (2) for the metered injection of a liquid, such as notably a reducing agent, characterized in that it consists in commanding openings of said injector for lengths of time that are suited to ensuring that the flow of liquid delivered upon each of the openings consists in a drop of liquid by commanding constant injector-opening times and adjusting the metered dose of liquid injected by varying the frequency of such openings.

2. The control method as claimed in claim 1, characterized in that injector (1) opening times substantially of the order of 1 millisecond are commanded.

3. The control method as claimed in claim 1, characterized in that when a motor vehicle stops, the procedure of injecting liquid drop by drop is continued for a predetermined period of time following this stopping.

4. The control method as claimed in claim 2, characterized in that when a motor vehicle stops, the procedure of injecting liquid drop by drop is continued for a predetermined period of time following this stopping.
